(19) United States
(12) Patent Application Publication
(10) Pub. No.: US 2024/0016482 A1
(43) Pub. Date: Jan. 18, 2024

(54) COMPACT BIOLOGICAL MATTER COLLECTION SYSTEMS

(71) Applicant: GYRUS ACMI, INC. D/B/A OLYMPUS SURGICAL TECHNOLOGIES AMERICA, Westborough, MA (US)

(72) Inventors: Nikhil M. Murdeshwar, Maple Grove, MN (US); Thomas J. Holman, Princeton, MN (US)

(21) Appl. No.: 18/032,111

(22) PCT Filed: Oct. 14, 2021
(86) PCT No.: PCT/US2021/071884

§ 371 Date: Apr. 14, 2023

Related U.S. Application Data
(60) Provisional application No. 63/092,659, filed on Oct. 16, 2020.

(51) Int. Cl. A61B 10/04; A61B 10/00; A61B 10/02

(52) U.S. Cl. CPC A61B 10/04; A61B 10/0096; A61B 10/0266

(57) ABSTRACT

A tissue separation device can comprise an elongate body comprising a proximal end portion and a distal end portion, a tissue separator coupled to the distal end portion, the tissue separator configured to engage sample tissue for retrieval, and a pressure-applying device configured to bias the tissue separator against the sample tissue. A method of collecting biological matter using a tissue retrieval device can comprise inserting the tissue retrieval device into anatomy of a patient, guiding a tissue collector of the tissue retrieval device to a target tissue area, activating a pressure applying device to bias the tissue collector into the target tissue, and collecting biological matter with the tissue retrieval device.

### CROSS-REFERENCE TO RELATED APPLICATION

This application claims the benefit of priority to U.S. Provisional Patent Application Ser. No. 63/092,659, filed Oct. 16, 2020, the contents of which are incorporated herein by reference in their entirety.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to medical devices that can be inserted into anatomy of a patient to perform a biological matter removal process, such as by cutting sample tissue for analysis.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path.

### SUMMARY

The present inventors have recognized that problems to be solved with conventional medical devices, and in particular endoscopes and duodenoscopes used to retrieve sample biological matter, include, among other things, 1) the difficulty in navigating endoscopes, and instruments inserted therein, to locations within anatomical regions deep within a patient, 2) the disadvantage of only being able to obtain small tissue sample sizes 3) the increased time and associated cost of having to repeatedly remove and reinsert medical devices to obtain a sufficient quantity of sample material, and 4), the difficulty of incorporating features (e.g., steerability and tissue collection features) into small-diameter devices. Such problems can be particularly present in duodenoscopy procedures (e.g., Endoscopic Retrograde Cholangio-Pancreatography, hereinafter “ERCP” procedures) where an auxiliary scope (also referred to as daughter scope, or cholangioscope) can be attached and advanced through the working channel of a “main scope” (also referred to as mother scope or duodenoscope). Furthermore, the tissue retrieval device used to remove the sample matter is inserted through the auxiliary scope. As such, the duodenoscope, auxiliary scope and tissue retrieval device become progressively smaller and more difficult to maneuver and perform interventions and treatments.

The present disclosure can help provide solutions to these and other problems by providing systems, devices and methods relating to inserting tissue retrieval devices, such as biopsy forceps, through a auxiliary scope having a small-diameter passage. The tissue retrieval devices can have a pressure-applying device that can be used to bias a tissue collector, e.g., a blade or auger, against target tissue. The pressure-applying device can facilitate the tissue collector removing a larger volume of sample material, such as by allowing the tissue collector to more deeply penetrate the target tissue. Furthermore, the tissue retrieval device can be used in conjunction with a receptacle that can hold one or more pieces of sample material, thereby allowing collection of multiple samples and larger samples. As such, the present disclosure can help solve the problems referenced above and other problems by 1) reducing the number of times a tissue retrieval device needs to be inserted and reinserted into the anatomy, and 2) increasing the volume of sample material collected with each insertion, among other things, as is described herein. The terms biological matter collection device, biological matter retrieval device, tissue collection device and tissue retrieval device are used interchangeably herein.

In an example, a tissue separation device can comprise an elongate body comprising a proximal end portion and a distal end portion, a tissue separator coupled to the distal end portion, the tissue separator configured to engage sample tissue for retrieval, and a pressure-applying device configured to bias the tissue separator against the sample tissue.

In another example, a method of collecting biological matter using a tissue retrieval device can comprise inserting the tissue retrieval device into anatomy of a patient, guiding a tissue collector of the tissue retrieval device to a target tissue area, activating a pressure applying device to bias the tissue collector into the target tissue, and collecting biological matter with the tissue retrieval device.

In an additional example, a tissue retrieval device can comprise an elongate shaft extending along an axis and configured for insertion into an anatomic duct, a tissue collection device coupled to the elongate shaft and configured to separate tissue from the anatomic duct, an energization device configured to improve engagement between the tissue collection device and the anatomic duct, and a control mechanism configured to selectively activate the energization device.

### BRIEF DESCRIPTION OF DRAWINGS

FIG. 1 is a schematic diagram of an endoscopy system comprising an imaging and control system and an endoscope, such as duodenoscope, with which the biological matter collection systems and devices of the present disclosure can be used.

FIG. 2 is a schematic diagram of the endoscopy system of FIG. 1 showing a schematic representation of the imaging and control system comprising a control unit connected to the endoscope.

FIG. 3A is a schematic top view of a distal portion of the endoscope of FIG. 2 comprising a camera module including optical components for a side-viewing endoscope and an elevator mechanism.

FIG. 3B is an enlarged cross-sectional view taken along the plane 3B-3B of FIG. 3A showing the optical components.

FIG. 3C is an enlarged cross-sectional view taken along the plane 3C-3C of FIG. 3A showing the elevator mechanism.

FIG. 4 is a schematic illustration of a distal portion of an endoscope being used to position an auxiliary scope proximate a duodenum, the auxiliary scope being configured to receive a biological matter collection device of the present disclosure.

FIG. 5A is a schematic illustration of a tissue retrieval device of the present disclosure comprising an elongate shaft, a tissue collector and a pressure-applying device in a retracted state.

FIG. 5B is a schematic illustration of the tissue collector with the pressure-applying device in a deployed configuration.

FIG. 6A is a schematic illustration of a tissue retrieval device comprising a scraping device and a spring-loaded actuator in a retracted state.

FIG. 6B is a schematic illustration of the tissue retrieval device of FIG. 6A with the spring-loaded actuator in a deployed state.

FIG. 7A is a top cross-sectional view of the tissue retrieval device of FIG. 6B taken along plane 7A-7A to show container space for the storage of collected matter.

FIG. 7B is a side cross-sectional view of the tissue retrieval device of FIG. 6A taken along plane 7B-7B to show a blade edge for the slicing of collected matter.

FIG. 7C is a side cross-sectional view of the tissue retrieval device of FIG. 6B taken along plane 7C-7C to show a sleeve positioned around the actuator and the container.

FIG. 8A is a schematic illustration of a tissue retrieval device comprising a boring device and an inflatable actuator in a collapsed state.

FIG. 8B is a schematic illustration of the tissue retrieval device of FIG. 8A with the inflatable actuator in an expanded state.

FIG. 9 is block diagram illustrating methods of collecting biological matter from a patient using the tissue retrieval devices of the present disclosure.

### DETAILED DESCRIPTION

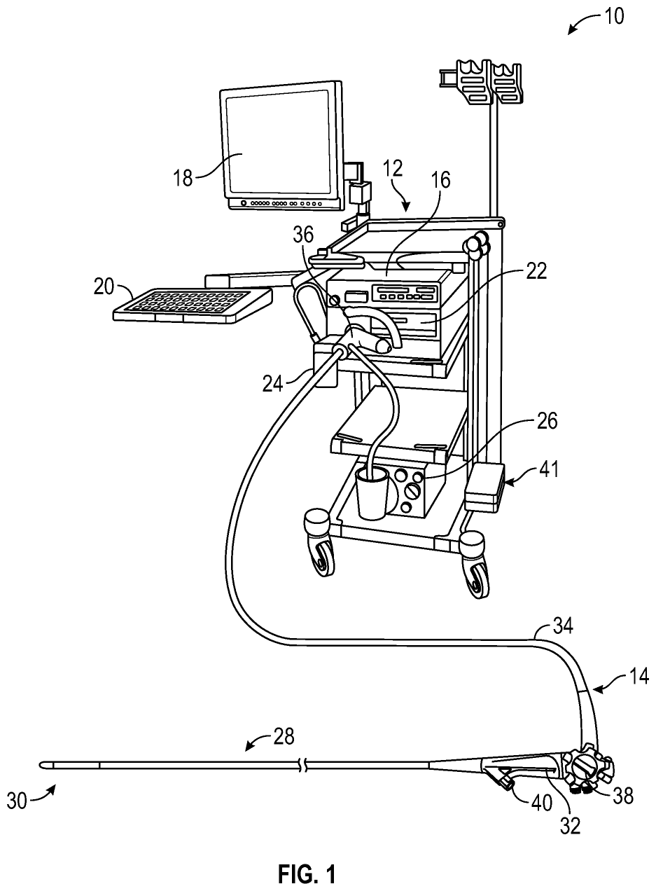

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as biological matter and tissue collection, retrieval and storage devices that can be used for obtaining samples of tissue or other biological matter to be removed from a patient for analysis or treatment of the patient. According to some examples, endoscope 14 can be insertable into an anatomical region for imaging and/or to provide passage of one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises a duodenoscope, though other types of endoscopes can be used with the features and teachings of the present disclosure.

Imaging and control system 12 can comprise controller 16, output unit 18, input unit 20, light source 22, fluid source 24 and suction pump 26.

Imaging and control system 12 can include various ports for coupling with endoscopy system 10. For example, controller 16 can include a data input/output port for receiving data from and communicating data to endoscope 14. Light source 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Controller 16 can additionally be used to generate signals or other outputs from treating the anatomical region into which endoscope 14 is inserted. In examples, controller 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36.

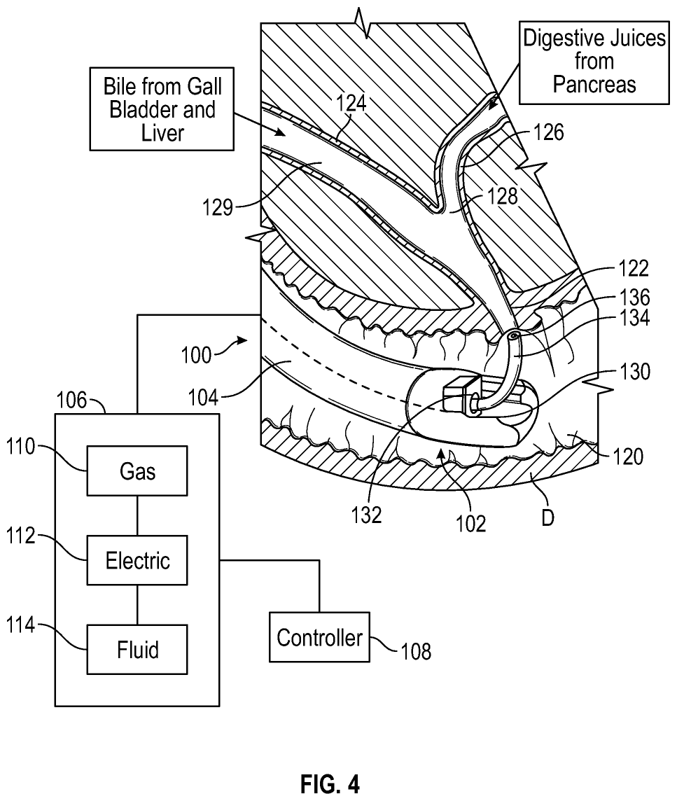

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as auxiliary scope 134 of FIG. 4. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise knob 38 as well as ports 40. Knob 38 can be coupled to a pull wire, or other actuation mechanisms, extending through insertion section 28. Ports 40 can be configured to couple various electrical cables, guide wires, auxiliary scopes, tissue collection devices of the present disclosure, fluid tubes and the like to handle section 32 for coupling with insertion section 28.

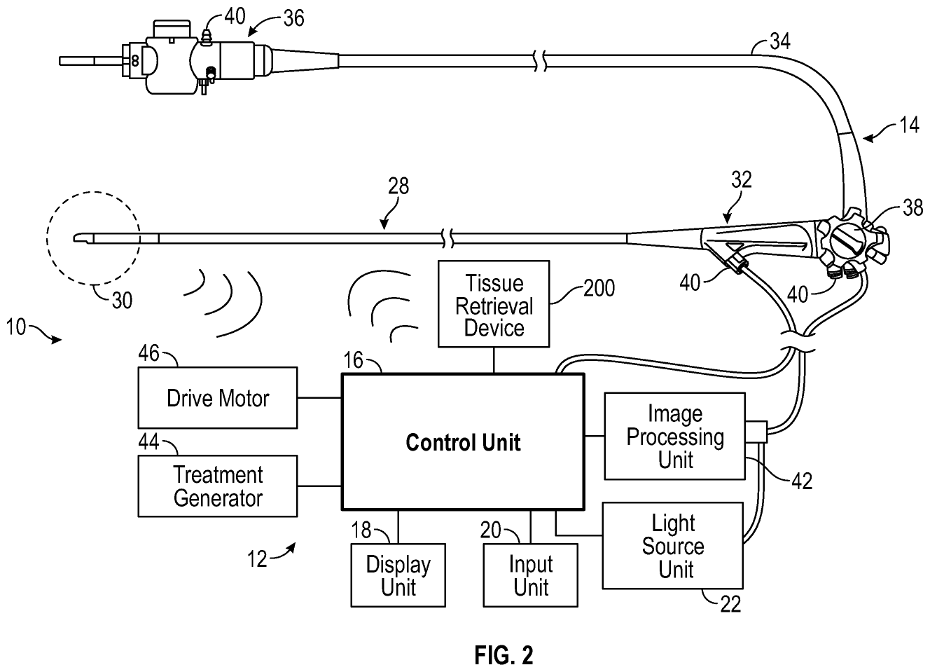

Imaging and control system 12, according to examples, can be provided on a mobile platform (e.g., cart 41) with shelves for housing light source 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

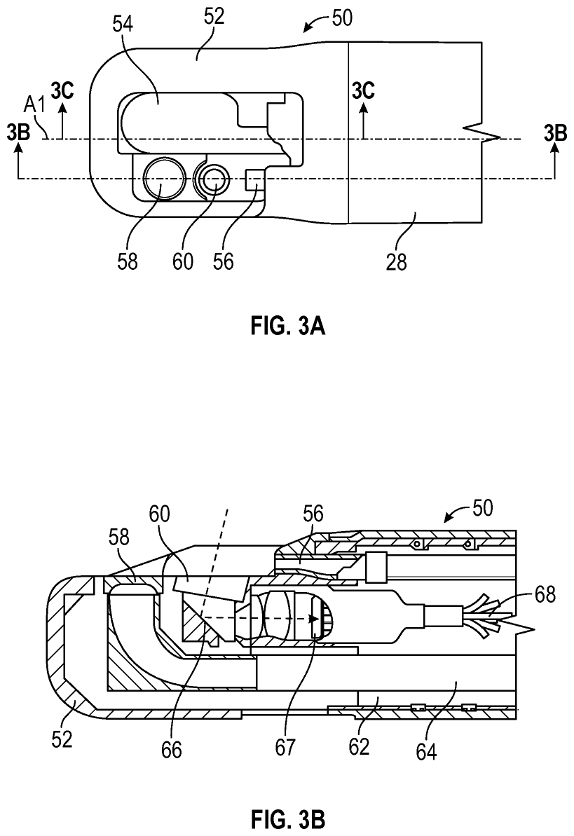

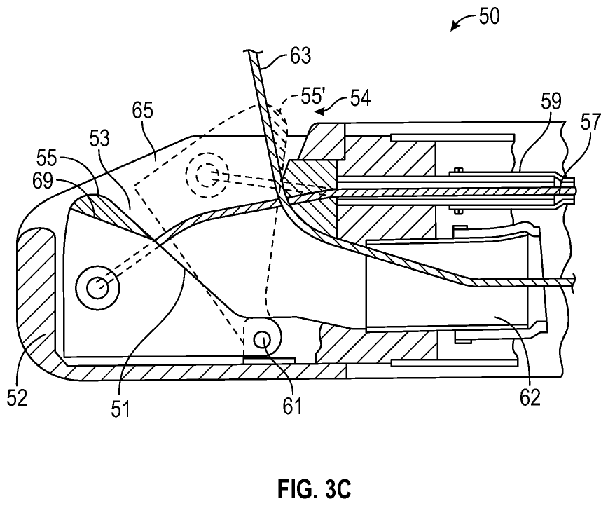

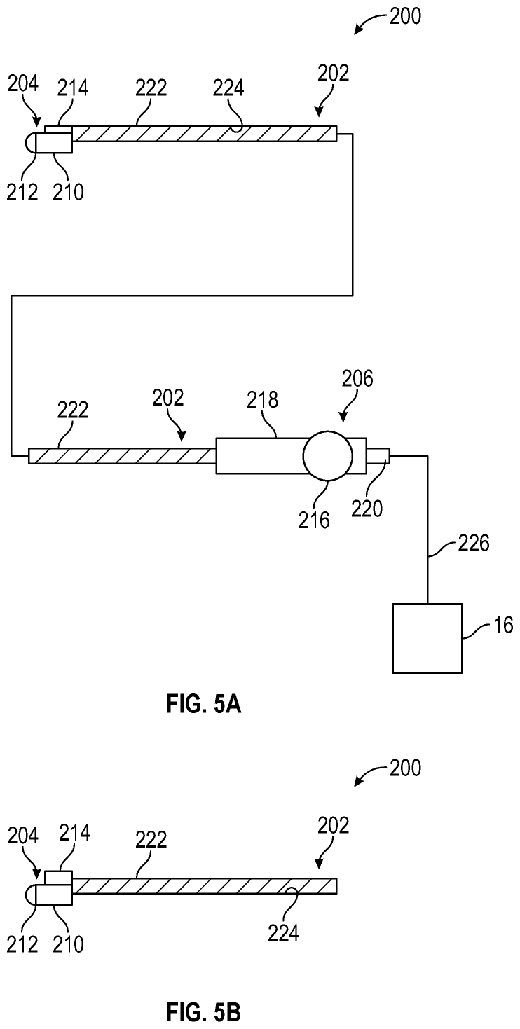

Functional section 30 can comprise components for treating and diagnosing anatomy of a patient. Functional section 30 can comprise an imaging device, an illumination device and an elevator, such as is described further with reference to elevator 54 of FIGS. 3A-3C. Functional section 30 can further comprise a biological matter collection system as is described herein. For example, functional section 30 can comprise one or more electrodes conductively connected to handle section 32 and functionally connected to imaging and control system 12 to analyze biological matter in contact with the electrodes based on comparative biological data stored in imaging and control system 12. In other examples, functional section 30 can directly incorporate a tissue collector similar to the tissue retrieval devices described with reference to FIGS. 5A-8B.

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise controller 16, which can include or be coupled to image processing unit 42, treatment generator 44 and drive unit 46, as well as light source 22, input unit 20 and output unit 18. As is discussed below in greater detail with reference to FIGS. 5A-8B, controller 16 can comprise, or can be in communication with, tissue retrieval device 134, which can comprise a device configured to press itself into tissue and collect and store a portion of that tissue. Controller 16 can be configured to activate an energization device (e.g., an actuator or a bladder) to bias or push a tissue collector retrieval device (e.g., a blade or an auger) against tissue to facilitate separation and the taking of larger samples.

Image processing unit 42 and light source 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with controller 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 3A-3C illustrate a first example of functional section 30 of endoscope 14 of FIG. 2. FIG. 3A illustrates a top view of functional section 30. FIG. 3B illustrates a cross-sectional view of functional section 30 taken along section plane 3B-3B of FIG. 3A. FIG. 3C illustrates a cross-sectional view of functional section 30 taken along section plane 3C-3C of FIG. 3A. FIGS. 3A-3C illustrate “side-viewing endoscope” (e.g., duodenoscope) camera module 50. In side-viewing endoscope camera module 50, illumination and imaging systems are positioned such that the viewing angle of the imaging system corresponds to a target anatomy lateral to central longitudinal axis A1 of endoscope 14. However, the biological matter retrieval devices can be used with other types of endoscopes, such as “end-viewing endoscopes.”

In the example of FIGS. 3A and 3B, side-viewing endoscope camera module 50 can comprise housing 52, elevator 54, fluid outlet 56, illumination lens 58 and objective lens 60. Housing 52 can form a fluid tight coupling with insertion section 28. Housing 52 can comprise opening for elevator 54. Elevator 54 can comprise a mechanism for moving a device inserted through insertion section 28, such as auxiliary scope 134 of FIG. 4. In particular, elevator 54 can comprise a device that can bend an elongate device extended through insertion section 28 along axis A1, as is discussed in greater detail with reference to FIG. 3C. Elevator 54 can be used to bend the elongate device at an angle to axis A1 to thereby treat or access the anatomical region adjacent side-viewing endoscope camera module 50. Elevator 54 is located alongside, e.g., radially outward of axis A1, illumination lens 58 and objective lens 60.

As can be seen in FIG. 3B, insertion section 28 can comprise central lumen 62 through which various components (e.g., auxiliary scope 134 (FIG. 4) can be extended to connect functional section 30 with handle section 32 (FIG. 2). For example, illumination lens 58 can be connected to light transmitter 64, which can comprise a fiber optic cable or cable bundle extending to light source 22 (FIG. 1). Likewise, objective lens 60 can be coupled to prism 66 and imaging unit 67, which can be coupled to wiring 68. Also, fluid outlet 56 can be coupled to fluid line 69, which can comprise a tube extending to fluid source 24 (FIG. 1). Other elongate elements, e.g., tubes, wires, cables, can extend through lumen 62 to connect functional section 30 with components of endoscopy system 10, such as suction pump 26 (FIG. 1) and treatment generator 44 (FIG. 2).

FIG. 3C a schematic cross-sectional view taken along section plane 3C-3C of FIG. 30 showing elevator 54. Elevator 54 can comprise deflector 55 that can be disposed in space 53 of housing 52. Deflector 55 can be connected to wire 57, which can extend through tube 59 to connect to handle section 32. Wire 57 can be actuated, such as by rotating a knob, pulling a lever, or pushing a button on handle section 32. Movement of wire 57 can cause rotation, e.g., clockwise, from a first position of deflector 55 about pin 61 to a second position of deflector 55, indicated by 55′. Deflector 55 can be actuated by wire 57 to move the distal portion of instrument 63 extending through window 65 in housing 52.

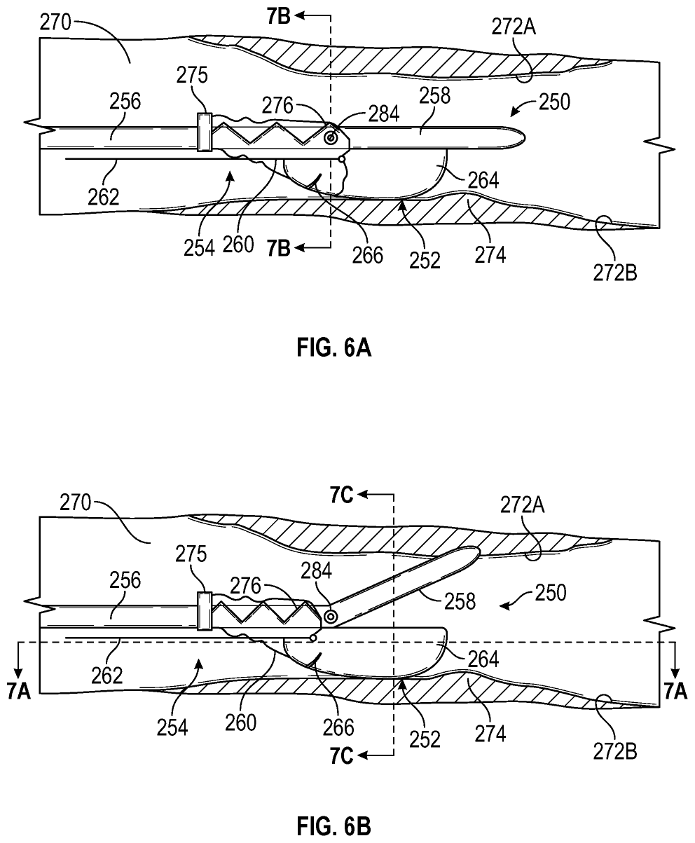

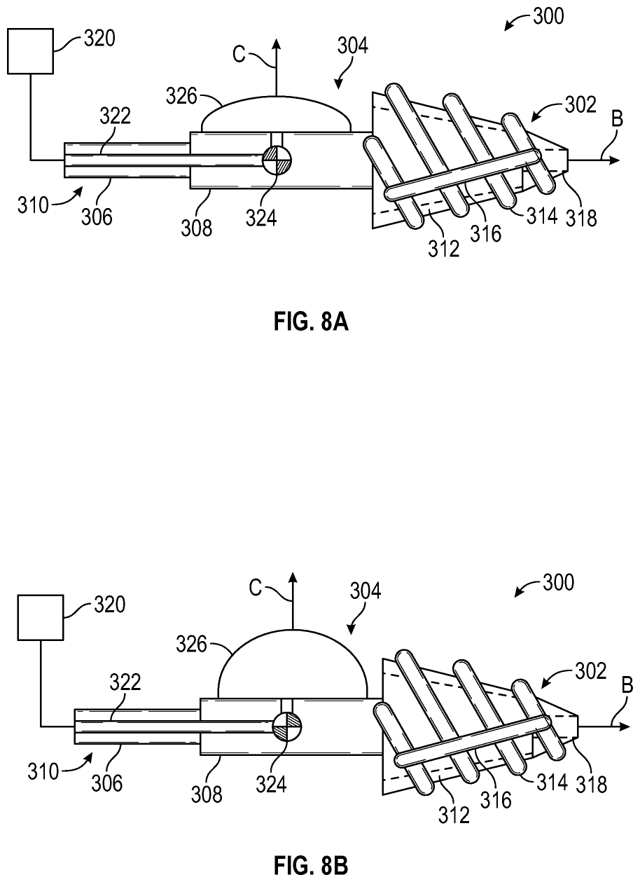

Housing 52 can comprise accommodation space 53 that houses deflector 55. Instrument 63 can comprise forceps, a guide wire, a catheter, or the like that extends through lumen 62. Instrument 63 can additionally comprise auxiliary scope 134 of FIG. 4, or a tissue collection device such as surgical instrument 200 of FIG. 5A, tissue retrieval device 250 (FIG. 6A) and tissue retrieval device 300 (FIG. 8A), as well as other instruments. A proximal end of deflector 55 can be attached to housing 62 at pin 61 provided to the rigid tip 21. A distal end of deflector 55 can be located below window 65 within housing 62 when deflector 55 is in the lowered, or un-actuated, state. The distal end of deflector 55 can at least partially extend out of window 65 when deflector 55 is raised, or actuated, by wire 57. Instrument 63 can slide on angled ramp surface 51 of deflector 55 to initially deflect the distal end of instrument 63 toward window 65. Angled ramp surface 51 can facilitate extension of the distal portion of instrument 63 extending from window 65 at a first angle relative to the axis of lumen 62. Angled ramp surface 51 can include groove 69, e.g. a v-notch, to receive and guide instrument 63. Deflector 55 can be actuated to bend instrument 63 at a second angle relative to the axis of lumen 62, which is closer to perpendicular that the first angle. When wire 57 is released, deflector 55 can be rotated, e.g., counter-clockwise, back to the lowered position, either by pushing or relaxing of wire 57. In examples, instrument 63 can comprise a cholangioscope or auxiliary scope 134 (FIG. 5).

Side-viewing endoscope camera module 50 of FIGS. 3A-3C can include optical components (e.g., objective lens 60, prism 66, imaging unit 67, wiring 68) for collection of image signals, lighting components (e.g., illumination lens 58, light transmitter 64) for transmission or generation of light. Endoscope camera module 50 can also include a photosensitive element, such as a charge-coupled device (“CCD” sensor) or a complementary metal-oxide semiconductor (“CMOS”) sensor. In either example, imaging unit 67 can be coupled (e.g., via wired or wireless connections) to image processing unit 42 (FIG. 2) to transmit signals from the photosensitive element representing images (e.g., video signals) to image processing unit 42, in turn to be displayed on a display such as output unit 18. In various examples, imaging and control system 12 and image processing unit 67 can be configured to provide outputs at desired resolution (e.g., at least 480p, at least 720p, at least 1080p, at least 4K UHD, etc.) suitable for endoscopy procedures.

Thus, as endoscope 100 is inserted further into the anatomy, the complexity with which it must be maneuvered and contorted increases, as described with reference to FIG. 4. Furthermore, in order to reach locations even further in the anatomy, additional devices can be used, e.g., instrument 63 in the form of auxiliary scope 134. As such, the cross-sectional area, e.g., diameter, of subsequently nested devices becomes smaller, thereby requiring even smaller devices that can be difficult to manufacture and manipulate, or satisfactorily produce results without repeated interventions (e.g., interactions with the patient), as is described with reference to FIGS. 5A-8B.

FIG. 4 is a schematic illustration of distal portion of endoscope 100 according to the present disclosure positioned in duodenum D. Endoscope 100 can comprise functional module 102, insertion section module 104, and control module 106. Control module 106 can include controller 108. Control module 106 can include other components, such as those described with reference to endoscopy system 10 (FIG. 1) and control unit 16 (FIG. 2). Additionally, control module 106 can comprise activation or energization components for applying pressure to the tissue retrieval devices described herein, such as air source 110, electric source 112 and liquid source 114. Endoscope 100 can be configured similarly as endoscope 14 of FIGS. 1 and 2.

Duodenum D can comprise duct wall 120, sphincter of Oddi 122, common bile duct 124 and main pancreatic duct 126. Duodenum D comprises an upper part of the small intestine. Common bile duct 124 carries bile from the gallbladder and liver (not illustrated) and empties the bile into the duodenum D through sphincter of Oddi 122. Main pancreatic duct 126 carries pancreatic juice from the exocrine pancreas (not illustrated) to common bile duct 124. Sometimes it can be desirable to remove biological matter, e.g., tissue, from bile duct 124 or pancreatic duct 126 to analyze the tissue to, for example, diagnose diseases or maladies of the patient such as cancer.

Functional module 102 can comprise elevator portion 130. Endoscope 100 can further comprise lumen 132 and auxiliary scope 134. Auxiliary scope 134 can comprise lumen 136. Though not shown for simplicity, auxiliary scope 134 can itself include functional components, such as a camera, to facilitate navigation of auxiliary scope 134 from endoscope 100 through the anatomy and to facilitate viewing of components extending from lumen 132.

In certain duodenoscopy procedures (e.g., Endoscopic Retrograde Cholangio-Pancreatography, hereinafter “ERCP” procedures) an auxiliary scope (also referred to as daughter scope, or cholangioscope), such as auxiliary scope 134, can be attached and advanced through lumen 132 (or central lumen 62 of insertion section 28 of endoscope 14 in FIG. 3B) of the “main scope” (also referred to as mother scope, or duodenoscope), such as endoscope 100. As discussed in greater detail below, auxiliary scope 134 can be guided into sphincter of Oddi 122. Therefrom, a surgeon operating auxiliary scope 134 can navigate auxiliary scope 134 through lumen 132 toward the gall bladder, liver or other locations in the gastrointestinal system to perform various procedures. The surgeon can navigate auxiliary scope 134 past entry 128 of main pancreatic duct 126 and into passage 129 of common bile duct 124, or into entry 128. Auxiliary scope 134 can be used to guide an additional device to the anatomy to obtain biological matter, such as by passage through lumen 136. The additional device can have its own functional devices, such as a light source, accessories, and biopsy channel, for therapeutic procedures. As described with reference to FIGS. 5A-8B, the additional device can include various features for gathering biological matter, such as tissue. The biological matter can then be removed from the patient, typically by removal of the additional device from the auxiliary device, so that the removed biological matter can be analyzed to diagnose one or more conditions of the patient. According to several examples, endoscope 100 can be suitable for the removal of cancerous or pre-cancerous matter (e.g., carcinoma, sarcoma, myeloma, leukemia, lymphoma and the like), endometriosis evaluation, biliary ductal biopsies, and the like.

However, as mentioned above, the size of the additional device is typically small due to the progressively smaller sizes of endoscope 100, auxiliary scope 134 and the additional device. In examples, lumen 132 of endoscope 100 can typically be on the order of approximately 4.0 mm in diameter, while lumen 136 of auxiliary scope 134 can typically be on the order of approximately 1.2 mm. As such, with conventional devices, it can be difficult to obtain sufficiently large tissue sample sized to ensure accurate diagnoses without having to repeatedly remove and reinsert the additional device. However, with the systems and devices of the present disclosure it is possible to obtain sufficiently large tissue sample sizes with only a single insertion and removal of the additional device, when configured as a tissue retrieval device of the present disclosure, for example.

FIG. 5A is a schematic illustration of surgical instrument 200 comprising elongate body 202, tissue collection device 204 and device controller 206. Surgical instrument 200 can comprise a device configured for the separation, collection and retrieval of biological matter, such as tissue, from a patient. Tissue collection device 204 can comprise container 210, separator 212, pressure-applying device 214 and activation mechanism 216. Controller 206 can comprise handpiece or handle 218, which can include activation mechanism 216 and connector 220. Elongate body 202 can comprise shaft 222 that can include lumen 224. System controller 206 can be connected to controller 16 (FIGS. 1 and 2) via cable 226 and the use of connector 220.

Tissue collection device 204 can be configured to do one or both of separate and retrieve biological matter from within a patient after being positioned within the patient by elongate body 202. Tissue collection device 204 can be configured to engage target tissue, aided by operation of pressure-applying device 214, separate the target tissue from the patient and store separated target tissue for removal from the patient, such as by removal of elongate body 202 from the patient.

Handpiece 218 can comprise any device suitable for facilitating manipulation and operation of surgical instrument 200. Handpiece 218 can be located at the proximal end of shaft 222 or another suitable location along shaft 222. In examples, handpiece 218 can comprise a pistol grip, a knob, a handlebar grip and the like. Actuation mechanism 216 can be attached to handpiece 218 to operate pressure-applying device 214. Actuation mechanism 216 can comprise one or more of buttons, triggers, levers, knobs, dials and the like. Actuation mechanism 216 can be coupled to pressure-applying device 214 and can comprise any suitable device for allowing operation of pressure-applying device 214 from handpiece 218. As such, actuation mechanism 216 can comprise a linkage located within lumen 224 of shaft 222 or alongside shaft 222. In examples, the linkage can be a mechanical linkage, an electronic linkage or an electric linkage, (such as a wire or cable), or an activation energy source, such as an electric source, a fluid source or a gas source (such as a tube or conduit).

Shaft 222 can extend from handpiece 218 and can comprise an elongate member configured to allow tissue collection device 204 to be inserted into a patient. In examples, shaft 222 can be sized for placement within an auxiliary scope, such as scope 134 of FIG. 4. As such, shaft 222 can be inserted into an incision in the epidermis of a patient, through a body cavity of the patient and into an organ. Thus, it is desirable for the diameter or cross-sectional shape of shaft 222, as well as components attached thereto, to be as small as possible to facilitate minimally invasive surgical procedures. Tissue collection device 204 can thus be incorporated into shaft 222 to minimize the size impact on surgical instrument 200 and without interfering with the linkage. Shaft 222 can be axially rigid, but resiliently bendable, and formed from a metal or plastic material.

Tissue collection device 204 can be located at the distal end of shaft 222 or another suitable location along shaft 222. Tissue collection device 204 can be sized to fit within lumen 136 (FIG. 4), for example. Tissue collection device 204 can comprise a component or device for interacting with a patient, such as those configured to cut, slice, pull, saw, punch, twist or auger tissue, and the like. Specifically, separator 212 can comprise any device suitable for removing tissue from a patient, such as a blade, punch, scraping device or an auger. In additional examples, separator 212 can comprise a device configured to scrape or abrade tissue from the patient, such as a brush or grater device. In another example, separator 212 can comprise a roughened surface, such as a surface coated with hard particles, such as diamond or sand particles. Separator 212 can be configured to physically separate portions of tissue of a patient from other larger portions of tissue in the patient. In additional examples, separator 212 can be configured to simply collect biological matter from the patient that does not need physical separation, such as mucus or fluid. In examples, separator 212 can be configured to physically separate portion of tissue of a patient for retrieval with the tissue collection device or another device. In examples, tissue collection device 204 can comprise container 210, separator 212, pressure-applying device 214 and activation mechanism 216.

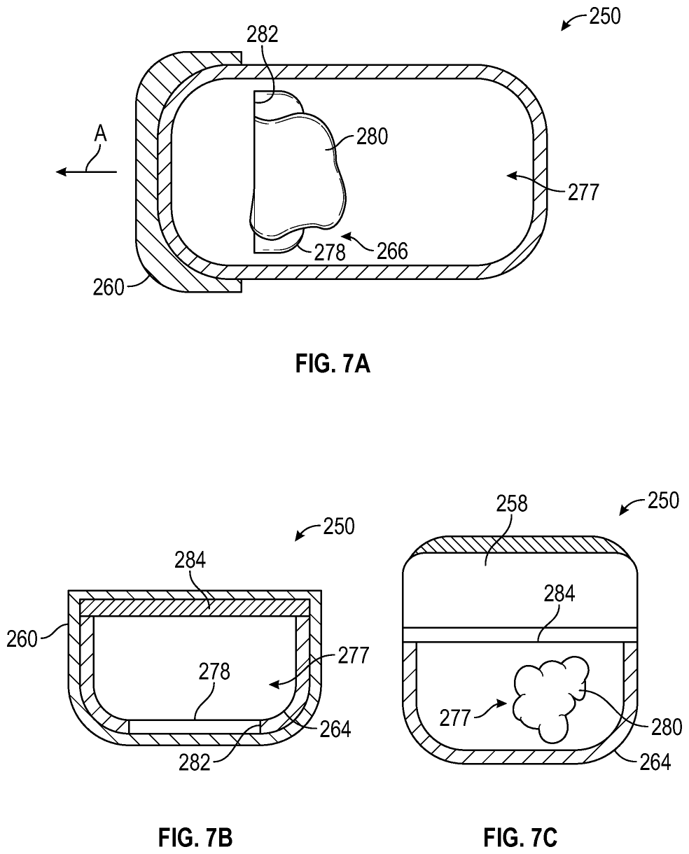

Pressure-applying device 214 can comprise a component or system that can be operated to selectively apply directional pressure to tissue collection device 204, either directly or through shaft 222. Pressure-applying device 214 can be coupled to shaft 222 or tissue collection device 204 in a position to push against biological structure opposite tissue collection device 204. As such, pressure-applying device 214 can facilitate engagement of separator 212 with target tissue. Pressure-applying device 214 can comprise any suitable device for pushing tissue collection device 204. In examples, pressure-applying device 214 can comprise a biasing element, such as a spring-loaded deflector, as is described with reference to FIGS. 6A-7C. In additional examples, pressure-applying device 214 can comprise an inflatable element, such as a balloon or bladder, as is described with reference to FIGS. 8A and 8B. Other pressure-applying forces can be used including magnetic repulsion forces.

Container 210 can comprise a walled element to hold and retain biological matter collected by tissue collection device 204. In an example, container 210 can comprise a flexible basket that can be deformed to allow separator 212 to be brought into close contact with target tissue. For example, container 210 can be fabricated from woven material such as strands of Kevlar, PVC, polyethylene, polycarbonate, PEEK and the like. Container 210 can be coupled to structural components, e.g., a frame, to facilitate coupling to shaft 222 and pressure-applying device 214, as well as to provide stability for separator 212. In additional examples, container 210 can comprise a structural element, such as a box fabricated from rigid and inflexible material.

Handpiece 218 can be operated by a user to operate tissue removal device 204. Handpiece 218 can be used to manipulate shaft 222 to push separator 212 against target tissue. For example, shaft 222 can be rotated, oscillated, reciprocated and the like move separator 212 along the target tissue to cause separator 212 to separate sample tissue from the target tissue attached to the patient. Activation mechanism 216 can be coupled to handpiece 218 and can be configured to operate pressure-applying device 214. Activation mechanism 216 can comprise any type of device suitable for activating the different types of pressure-applying devices described herein. In examples, activation mechanism 216 can comprise one or more of a lever, a trigger, a joystick, a button, a wheel and the like, as well as combinations thereof. In an example, activation mechanism 216 can comprise a wheel that can be rotated in one direction to activate or energize a pressure-applying mechanism and rotated in an opposite direction to deactivate or deenergize the pressure-applying mechanism. For example, the wheel can be rotated to push and/or pull a wire or open and close a valve. As shown in FIG. 5B, activation mechanism 216 can be engaged to activate pressure-applying device such that, for example, the size, geometry or position of pressure-applying device can be changed to push against an anatomical surface and cause a corresponding reactive force to be applied to separator 212.

As mentioned above and discussed in further detail below, tissue removal device 204 can be configured as a low-profile device so as to be able to be inserted through a small diameter lumen, such as lumen 136 of auxiliary scope 134 of FIG. 4. Additionally, tissue removal device 204 can be configured as a high-capacity tissue collector that can hold a large volume of collected sample tissue to thereby reduce or eliminate the need to repeatedly remove surgical instrument 200 from the auxiliary scope.

FIG. 6A is a schematic illustration of tissue retrieval device 250 comprising scraping device 252 and spring-loaded actuator 254 in a retracted state. FIG. 6B is a schematic illustration of tissue retrieval device 250 with spring-loaded actuator 254 in a deployed state. In the illustrated example, tissue retrieval device 250 and actuator 254 can be mounted to shaft 256, proximate a distal end of shaft 256. Actuator 254 can comprise projection 258, sleeve 260 and pull-cord 262. Scraping device 250 can comprise container 264 and blade 266.

As shown in FIG. 6A, tissue retrieval device 250 can be positioned in anatomic duct 270, which can comprise first wall portion 272A and second wall portion 272B. Second wall portion 272B can comprise target tissue 274. Shaft 256 can be used to guide scraping device 252 through anatomic duct 270 to target tissue 274. Target tissue 274 can comprise a protrusion, such as a growth of cancerous or pre-cancerous material.

Tissue retrieval device 250 can be inserted into anatomic duct 270 with sleeve 260 in a distal or disengaged position, as shown in FIG. 6A. Sleeve 260 can be retracted proximally using pull-cord 262. Sleeve 260 can be anchored to shaft 256 using band 275, which can be axially fixed relative to shaft 256. Pull-cord 262 can be coupled to a distal end portion of sleeve 260, such as by using a mechanical fastener, a chemical bond or an adhesive, etc. Sleeve 260 can comprise a piece of material that surrounds, or at least partially surrounds tissue retrieval device 250. Sleeve 260 can be pliable so as to be furled between band 275 and the distal end portion of sleeve 260. Sleeve 260 can incorporate, or otherwise be configured to operate with, spring 276, or another biasing element, that can be used to bias the distal end portion of sleeve 260 distally away from band 275. Thus, pull-cord 262 can be drawn proximally to retract sleeve 260 and expose blade 266 and thereby furrow sleeve 260 out of the way of blade 266. Pull-cord 262 can be released so that spring 276 can push the distal end portion of sleeve 260 distally to cover blade 266. In other examples, sleeve 260 and pull-cord 262 can comprise rigid bodies, with band 275 and spring 276 omitted. As such, sleeve 260 can be slid along shaft proximally and distally by puling and pushing of pull-cord 262.

FIG. 7A is a top cross-sectional view of tissue retrieval device 250 of FIGS. 6A and 6B showing space 277 within container 264 for the storage of collected matter. As shown in FIG. 7A, sleeve 260 can be retracted proximally in the direction of arrow A. For example, pull-cord 262 can be pulled proximally to pull sleeve 260 away from blade 266. With pressure-applying device 250 activated, blade edge 278 can be pressed against target tissue 274 (FIG. 6A). Tissue retrieval device 250 can be moved in the direction of arrow A, such as by pulling on shaft 256 (FIG. 6A) by a user, to cause sample tissue 280 to move into container 264. Tissue retrieval device 250 can be reciprocated back-and-forth along the axis of arrow A to collect additional pieces of sample tissue 280.

FIG. 7B is a side cross-sectional view of tissue retrieval device 250 of FIGS. 6A and 6B showing blade edge 278 of blade 266 for the slicing of biological matter to be collected. Blade 266 can comprise a device configured to simultaneously separate tissue from anatomic duct 270 and direct separated tissue into space 277 of container 264. Blade edge 278 can be fabricated out of an edge of opening 282 in container 264. In examples, blade edge 278 can be curved into or out of container 264 to facilitate engagement with and slicing of target tissue 274 (FIG. 6A). In examples, blade 266 can be configured similarly to a potato peeler.

As shown in FIG. 7B, sleeve 260 can be configured to cover blade 266 in a non-retracted or distally-positioned state. As such, tissue retrieval device 250 can be configured to be inserted into a patient, e.g., distally into anatomic duct 270 (in the opposite direction of where arrow A is pointing), without unintentionally engaging anatomic duct 270. Furthermore, in the non-retracted state, sleeve 260 can be configured to push actuator 258 radially inward, relative to the axis of shaft 256, toward container 264. In the illustrated example, sleeve 260 can push actuator 258 against container 264 to thereby retain sample tissue 280 therein.

FIG. 7C is a side cross-sectional view of tissue retrieval device 250 of FIGS. 6A and 6B showing sleeve 260 positioned around actuator 256 and container 264. Actuator 256 can comprise biasing element 284, such as a spring, to cause projection 258 to move away from container 264. Projection 258 can be configured to push scraping device 252 perpendicular to the axis of shaft 256. However, due to rotation of actuator 256 about biasing element 284, projection 258 can be configured to provide axial biasing to scraping device 252. The length of projection 258 can be configured to provide a desired amount of pressure for scraping device 252, with longer projections 258 being configured to apply more force. Thus, projection 258 can extend beyond the distal end of container 264. Tissue retrieval device 250 can be configured in multiple models or configurations with different sized (e.g., lengths) projections 258 for use in different sized body cavities.

FIG. 8A is a schematic illustration of tissue retrieval device 300 comprising boring device 302 and inflatable actuator 304 in a collapsed state. FIG. 8B is a schematic illustration of tissue retrieval device 300 comprising boring device 302 and inflatable actuator 304 in an expanded state. FIG. 8A and 8B are discussed concurrently.

Tissue retrieval device 300 can further comprise shaft 306, sleeve 308 and energization system 310. Boring device 302 can comprise container 312, boring lands 314, blade 316 and bore 318. Energization system 310 can comprise energy source 320, duct 322 and valve 324. Actuator 304 can comprise bladder 326.

Tissue retrieval device 300 can be configured to engage tissue in the axial direction of arrow B. For example, tissue retrieval device 300 can be positioned in front of a mound or protrusion of tissue (e.g., target tissue 274 of FIG. 6A) or proximate a wall of tissue (e.g., duct 270 of FIG. 6A). Shaft 306 can be advanced in the direction of arrow A by a user to engage the target tissue. In some situations, it is possible for boring device 302 to slip over the target tissue, such as due to slippery or moist conditions. Thus, it can be difficult or impossible to engage the tissue sufficiently to collect a desirable volume of sample tissue. Inflatable actuator 304 can be employed to expand in the direction of arrow C, as shown in FIG. 8B, thereby pushing boring device 302 perpendicularly toward the axis of arrow B. As such, as boring device 302 is advanced forward, the distal tip of container 312 can maintain engagement with the tissue.

Inflatable actuator 304 can comprise a pressure-applying device configured to be energized with a fluid or gas. Energy source 320 can comprise a source of pressurized fluid, such as air or saline. The pressurized fluid can flow from energy source 320 to bladder 326 through duct 322. Valve 324 can be positioned in duct 322 to selectively allow the pressurized fluid into the internal cavity of bladder 326. Valve 324 can be mechanically or electrically activated and can be controlled by an actuator connected to a handpiece (e.g., handpiece 218 of FIG. 5A) located on shaft 306, or an actuator located on controller 16 (FIG. 1). In other example, valve 324 can be located at a proximal location along shaft 306, such as to be positioned to not be inserted into the patient.

In examples, boring device 302 can be configured as an auger. As such, container 312 can have a cone shape with lands 314 wrapped around container 312 in a spiral manner. Lands 314 can be configured to engage tissue to allow container 312 to penetrate the tissue in the direction of arrow B. Shaft 306 can be rotated by an operator to rotate container 312 and lands 314. Lands 314 can grab tissue while being rotated to cause further axial penetration of boring device 302 into the tissue. Bladder 326 can be mounted on sleeve 308 through which shaft 306 can pass to allow shaft 306 to rotate boring device 302 without affecting the directionality of inflatable actuator 304. As container 312 enters tissue, blade 316 can be configured to slice or shave tissue away from the patient. Blade 316 can comprise a sharpened edge of an opening in container 312 and can be configured similar to a potato peeler as discussed herein. Additionally, container 312 can include distal bore 318 that can be configured to punch through tissue to take a tissue sample similar to core sampling a tree, etc. As such, the distal or leading edge of bore 318 can be sharpened. In examples, only one of blade 316 and bore 318 can be used. In other examples, boring device 302 can be configured to simply punch into the tissue such that tissue enters bore 318. Thus, boring device 302 can be configured as a punch. In such a configuration, lands 314 and blade 316 can be omitted from container 312. In the various examples, container 312 can be configured to have an internal space to capture and retain sample tissue collected by bore 318 and/or blade 316.

In another example, inflatable actuator can be configured to be activated by magnetic repulsion. A first magnet can be attached to bladder 326 and a second magnet can be attached to sleeve 308. Sleeve 308 can be rotated to radially align the first and second magnets to push bladder 326 away from shaft 306. Sleeve 308 can be rotated to un-align the first and second magnets to allow bladder 326 to fall back toward shaft 306. In another example, first and second magnets can be stationarily aligned and can be electromechanically energized to produce a magnetic field.

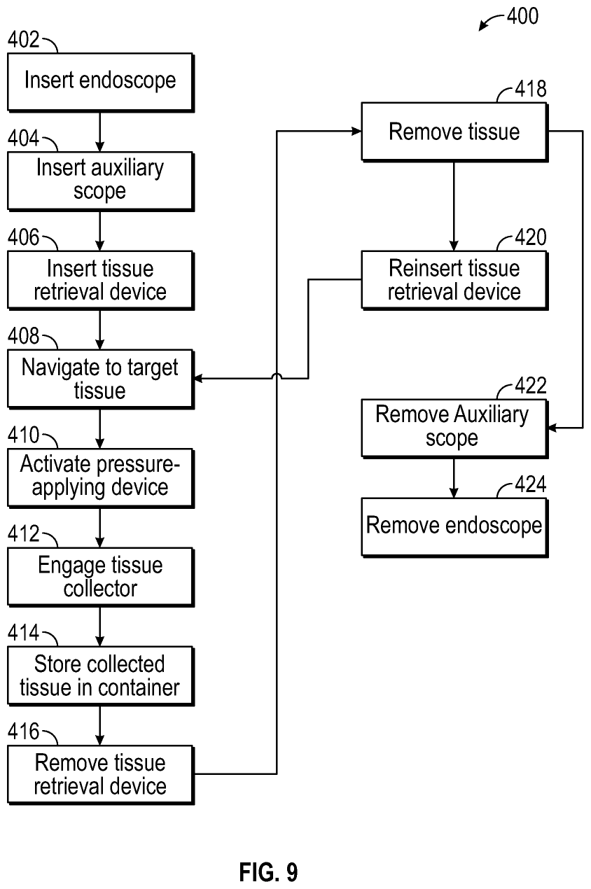

FIG. 9 is block diagram illustrating examples of method 400 of collecting biological matter from a patient using the tissue retrieval devices of the present disclosure. Method 400 can encompass the use of surgical instrument 200 of FIG. 5A, tissue retrieval device 250 (FIG. 6A) and tissue retrieval device 300 (FIG. 8A), as well as other instruments.

At step 402, an endoscope can be inserted into and navigated through anatomy of a patient. For example, endoscope 14 (FIG. 1) can utilize native imaging capabilities to guide insertion section 28 through anatomic ducts of the patient. Insertion section 28 can be bent or curved using control knob 38 to facilitate turning of endoscope 14.

At step 404, an auxiliary scope can be inserted into the endoscope to access anatomy located further in the duct. For example, auxiliary scope 134 (FIG. 4) can be inserted into lumen 62 (FIG. 3C) or lumen 132 (FIG. 4) to reach another anatomic duct intersecting the anatomic duct reached by endoscope 14. Elevator 54 (FIG. 3C) can be used to bend or turn auxiliary scope 134.

At step 406, a tissue retrieval device can be inserted into the auxiliary scope to reach target tissue distal of the auxiliary scope. For example, surgical instrument 200 (FIG. 5A) can be inserted such that tissue collection device 204 extends beyond the distal end of auxiliary scope 134.

At step 408, the tissue collection device can be navigated to the location of target tissue within the patient. For example, tissue collection device 204 can be navigated through an anatomic duct to target tissue 274 (FIG. 6A). The target tissue can comprise tissue that is potentially diseased or otherwise indicative of a diseased condition of the patient.

At step 410, a pressure-applying device can be activated so as to push the tissue collection device against target tissue. For example, pressure-applying device 214 (FIG. 5A) can be actuated or energized using activation mechanism 216. For example, activation mechanism 216 (FIG. 6A) can be rotated to wind pull-cord 262 to retract sleeve 260 away from projection 258, thereby causing projection 258 to push against tissue opposing target tissue 274.

At step 412, a tissue collection device can be pushed, pressed or otherwise brought into pressurized contact with the target tissue. For example, tissue collection device 204 can be pushed by pressure-applying device 214 into target tissue 274. Thus, tissue collection device 204 can be reciprocated axially, or rotated, to cause blade 266 to slice, punch or shave, etc. one or more pieces of tissue away from the anatomy of the patient.

At step 414, sample tissue or biological matter separated or collected from the patient at step 412 can be stored within a space inside the tissue collection device. For example, as tissue collection device 204 is manipulated back-and-forth, or rotated, separated sample tissue 280 can slide past blade edge 278 into space 277 of container 264.

At step 416, the tissue collection device can be removed from the patient, such as by removal from the auxiliary scope, which can be left in place inside the anatomy. Safeguards can be put into place to ensure removal of the tissue collection device without inadvertently cutting anatomy of the patient. For example, sleeve 260 can be repositioned around housing 264 to cover blade 266 and retract projection 258.

At step 418, the collected sample tissue can be removed from the tissue collection device. For example, projection 258 can be rotated away from container 264 to allow user access to space 277 so that sample tissue 280 can be removed for analysis, etc.

Thereafter, method 400 can return to step 408 or can continue to step 420.

At step 420, the tissue collection device can be reinserted. From step 420, steps 408 to 418 can be repeated as many times as desired to achieve a suitable amount of sample tissue, such as a quantity sufficient to perform laboratory testing to ascertain a diagnosis to a high level of certainty. Note that the present disclosure is directed to systems and methods that reduce or eliminate the need to reinsert tissue retrieval devices. However, in some cases it may be desirable to do so in order to collect additional sample material from the same site or to collect sample material from a different site.

At step 422, the auxiliary scope can be removed from the endoscope.

At step 424, the endoscope can be removed from the patient.

As such, method 400 illustrates examples of a method of collecting biological matter from internal passages of a patient in large enough quantities, e.g., by using a directionally enhanced tissue removal device with internal storage, to eliminate or reduce insertion and removal of surgical devices from the patient.

### VARIOUS NOTES & EXAMPLES

Example 1 can include or use subject matter such as a tissue separation device comprising an elongate body comprising a proximal end portion and a distal end portion, a tissue separator coupled to the distal end portion, the tissue separator configured to engage sample tissue for retrieval by the tissue separator; and a pressure-applying device configured to bias the tissue separator against the sample tissue.

Example 2 can include, or can optionally be combined with the subject matter of Example 1, to optionally include a release mechanism to selectively activate the pressure-applying device from the proximal end portion of the elongate body.

Example 3 can include, or can optionally be combined with the subject matter of one or any combination of Examples 1 or 2 to optionally include a container to receive tissue retrieved by the tissue separator.

Example 4 can include, or can optionally be combined with the subject matter of one or any combination of Examples 1 through 3 to optionally include a tissue separator comprising a scraping device.

Example 5 can include, or can optionally be combined with the subject matter of one or any combination of Examples 1 through 4 to optionally include a scraping device comprising a blade.

Example 6 can include, or can optionally be combined with the subject matter of one or any combination of Examples 1 through 5 to optionally include a container comprises an opening, and a blade disposed along an edge of the opening.

Example 7 can include, or can optionally be combined with the subject matter of one or any combination of Examples 1 through 6 to optionally include a blade that is configured to cut tissue when the scraping device moves in a direction extending from the distal end toward the proximal end.

Example 8 can include, or can optionally be combined with the subject matter of one or any combination of Examples 1 through 3 to optionally include a tissue separator comprises a penetrating device.

Example 9 can include, or can optionally be combined with the subject matter of one or any combination of Examples 1 through 3 and 8 to optionally include a penetrating device comprises an auger.

Example 10 can include, or can optionally be combined with the subject matter of one or any combination of Examples 8 to 9 to optionally include a container comprising an opening, and an auger that wraps around the opening.

Example 11 can include, or can optionally be combined with the subject matter of one or any combination of Examples 8 through 10 to optionally include an auger that is configured to cut tissue when the tissue separator is rotated about a central axis.

Example 12 can include, or can optionally be combined with the subject matter of one or any combination of Examples 1 through 11 to optionally include a pressure-applying device comprising a biased actuator.

Example 13 can include, or can optionally be combined with the subject matter of one or any combination of Examples 1 through 12 to optionally include a spring-loaded actuator comprising a projecting member connected to the container at a pivot point, and a spring configured to bias the projecting member away from the housing.

Example 14 can include, or can optionally be combined with the subject matter of one or any combination of Examples 1 through 13 to optionally include a release mechanism comprising a sleeve configured to slide along the elongate body between a distal position and a proximal position, and a pull-cord connected to the sleeve and configured to extend along the elongate body to the proximal end, wherein in the distal position the sleeve is configured to press the projecting member against the tissue collector, and wherein the pull-cord is configured to move the sleeve to the proximal position to allow the projecting member to pivot away from the tissue collector.

Example 15 can include, or can optionally be combined with the subject matter of one or any combination of Examples 1 through 11 to optionally include a pressure-applying device comprising an expandable body.

Example 16 can include, or can optionally be combined with the subject matter of one or any combination of Examples 1 through 11 and 15 to optionally include an expandable body comprise an inflatable bladder.

Example 17 can include, or can optionally be combined with the subject matter of one or any combination of Examples 15 to 16 to optionally include a release mechanism that comprises a valve connected to the inflatable bladder, and a fluid tube extending from the valve.

Example 18 can include, or can optionally be combined with the subject matter of one or any combination of Examples 15 through 17 to optionally include a shield configured to selectively cover the tissue separator.

Example 19 can include, or can optionally be combined with the subject matter of one or any combination of Examples 3 through 18 to optionally include a container that is resiliently flexible.

Example 20 can include, or can optionally be combined with the subject matter of one or any combination of Examples 1 through 19 to optionally include an endoscope comprising a first longitudinal passage, and an auxiliary scope comprising a second longitudinal passage, where the auxiliary scope is configured to slide in the first longitudinal passage, wherein the tissue separation device is configured to slide in the second longitudinal passage.

Example 21 can include or use subject matter such as a method of collecting biological matter using a tissue retrieval device that can comprise inserting the tissue retrieval device into anatomy of a patient, guiding a tissue collector of the tissue retrieval device to a target tissue area, activating a pressure applying device to bias the tissue collector into the target tissue, and collecting biological matter with the tissue retrieval device.

Example 22 can include, or can optionally be combined with the subject matter of Example 21, to optionally include inserting the tissue retrieval device into the anatomy by extending an elongate shaft into a duct of the anatomy.

Example 23 can include, or can optionally be combined with the subject matter of one or any combination of Examples 21 or 22 to optionally include collecting biological matter with the tissue retrieval device by scraping the tissue retrieval device along the target tissue area.

Example 24 can include, or can optionally be combined with the subject matter of one or any combination of Examples 21 through 23 to optionally include collecting biological matter with the tissue retrieval device by advancing the tissue retrieval device in a direction to withdraw the tissue retrieval device from the anatomy.

Example 25 can include, or can optionally be combined with the subject matter of one or any combination of Examples 21 through 24 to optionally include collecting biological matter with the tissue retrieval device comprises pushing the tissue retrieval device into the target tissue area.

Example 26 can include, or can optionally be combined with the subject matter of one or any combination of Examples 21 through 25 to optionally include pushing the tissue retrieval device into the target tissue area comprises rotating an auger comprising the tissue retrieval device.

Example 27 can include, or can optionally be combined with the subject matter of one or any combination of Examples 21 through 26 to optionally include activating the pressure applying device to bias the tissue collector into the target tissue by releasing a spring-loaded deflector.

Example 28 can include, or can optionally be combined with the subject matter of one or any combination of Examples 21 through 27 to optionally include releasing the spring-loaded deflector by retracting a sleeve from around the spring-loaded deflector.

Example 29 can include, or can optionally be combined with the subject matter of one or any combination of Examples 21 through 28 to optionally include activating the pressure applying device to bias the tissue collector into the target tissue by expanding a body.

Example 30 can include, or can optionally be combined with the subject matter of one or any combination of Examples 21 through 29 to optionally include expanding the body by inflating a bladder.

Example 31 can include, or can optionally be combined with the subject matter of one or any combination of Examples 21 through 30 to optionally include inserting an endoscope comprising a first longitudinal passage into the anatomy of the patient, inserting an auxiliary scope comprising a second longitudinal passage into the first longitudinal passage, and inserting the tissue retrieval device into the second longitudinal passage to reach the target tissue.

Example 32 can include or use subject matter such as a tissue retrieval device that can comprise an elongate shaft extending along an axis and configured for insertion into an anatomic duct, a tissue collection device coupled to the elongate shaft and configured to separate tissue from the anatomic duct, an energization device configured to improve engagement between the tissue collection device and the anatomic duct, and a control mechanism configured to selectively activate the energization device.

Example 33 can include, or can optionally be combined with the subject matter of Example 32, to optionally include a shield to selectively prevent the tissue collection device from separating tissue from the anatomic duct.

Example 34 can include, or can optionally be combined with the subject matter of one or any combination of Examples 32 or 33 to optionally include a container having an interior space in communication with the tissue collection device.

Example 35 can include, or can optionally be combined with the subject matter of one or any combination of Examples 32 through 34 to optionally include an energization device that utilizes fluid pressure.

Example 36 can include, or can optionally be combined with the subject matter of one or any combination of Examples 32 through 35 to optionally include an energization device that comprises a bladder.

Example 37 can include, or can optionally be combined with the subject matter of one or any combination of Examples 32 through 36 to optionally include an energization device utilizes mechanical pressure.

Example 38 can include, or can optionally be combined with the subject matter of one or any combination of Examples 32 through 37 to optionally include an energization device that comprises a spring-loaded detent.

Example 39 can include, or can optionally be combined with the subject matter of one or any combination of Examples 32 through 38 to optionally include a tissue collection device that is configured to slice tissue from the anatomic duct in an axial direction.

Example 40 can include, or can optionally be combined with the subject matter of one or any combination of Examples 32 through 39 to optionally include a tissue collection device that is configured to bore tissue from the anatomic duct in an axial direction.

Example 41 can include, or can optionally be combined with the subject matter of one or any combination of Examples 32 through 42 to optionally include a tissue collection device that is configured to slice tissue from the anatomic duct in a circumferential direction.

Example 42 can include, or can optionally be combined with the subject matter of one or any combination of Examples 32 through 41 to optionally include a tissue collection device that is rotatable relative to the elongate shaft.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A tissue separation device comprising:
an elongate body comprising:
a proximal end portion; and
a distal end portion;
a tissue separator coupled to the distal end portion, the tissue separator configured to engage sample tissue for retrieval; and
a pressure-applying device coupled to one or both of the elongate body and the tissue separator and that is_configured to bias the tissue separator against the sample tissue.

2. The tissue separation device of claim 1, further comprising a release mechanism to selectively activate the pressure-applying device from the proximal end portion of the elongate body.

3. The tissue separation device of claim 2, further comprising a container to receive tissue retrieved by the tissue separator.

4. The tissue separation device of claim 3, wherein the tissue separator comprises a scraping device.

5. The tissue separation device of claim 4, wherein the scraping device comprises a blade, wherein:
the container comprises an opening; and
the blade is disposed along an edge of the opening.

6. (canceled)

7. The tissue separation device of claim 4, wherein the blade is configured to cut tissue when the scraping device moves in a direction extending from the distal end toward the proximal end.

8. The tissue separation device of claim 3, wherein the tissue separator comprises a penetrating device.

9. The tissue separation device of claim 8, wherein the penetrating device comprises an auger.

10. The tissue separation device of claim 9, wherein:
the container comprises an opening; and
the auger wraps around the opening,.
wherein the auger is configured to cut tissue when the tissue separator is rotated about a central axis.

11. (canceled)

12. The tissue separation device of claim 3, wherein the pressure-applying device comprises a biased actuator.

13. The tissue separation device of claim 12, wherein the biased actuator comprises:
a projecting member connected to the container at a pivot point; and
a spring configured to bias the projecting member away from the housing.

14. The tissue separation device of claim 13, wherein the release mechanism comprises:
a sleeve configured to slide along the elongate body between a distal position and a proximal position; and
a pull-cord connected to the sleeve and configured to extend along the elongate body to the proximal end;
wherein in the distal position the sleeve is configured to press the projecting member against the tissue collector; and
wherein the pull-cord is configured to move the sleeve to the proximal position to allow the projecting member to pivot away from the tissue collector.

15. The tissue separation device of claim 3, wherein the pressure-applying device comprises an expandable body.

16. The tissue separation device of claim 15, wherein the expandable body comprise an inflatable bladder.

17. The tissue separation device of claim 16, wherein the release mechanism comprises:
a valve connected to the inflatable bladder; and
a fluid tube extending from the valve.

18. The tissue separation device of claim 3, further comprising a shield configured to selectively cover the tissue separator.

19. The tissue separation device of claim 3, wherein the container is resiliently flexible.

20. The tissue separation device of claim 1, further comprising:
an endoscope comprising a first longitudinal passage; and
an auxiliary scope comprising a second longitudinal passage, where the auxiliary scope is configured to slide in the first longitudinal passage;
wherein the tissue separation device is configured to slide in the second longitudinal passage.

21. A method of collecting biological matter using a tissue retrieval device, the method comprising:
inserting the tissue retrieval device into anatomy of a patient;
guiding a tissue collector of the tissue retrieval device to a target tissue area;
activating a pressure applying device to bias the tissue collector into the target tissue; and
collecting biological matter with the tissue retrieval device.

22. The method of claim 21, wherein inserting the tissue retrieval device into the anatomy comprises extending an elongate shaft into a duct of the anatomy.

23. The method of claim 21, wherein collecting biological matter with the tissue retrieval device comprises scraping the tissue retrieval device along the target tissue area and advancing the tissue retrieval device in a direction to withdraw the tissue retrieval device from the anatomy.

24. (canceled)

25. The method of claim 21, wherein:
collecting biological matter with the tissue retrieval device comprises pushing the tissue retrieval device into the target tissue area; and
pushing the tissue retrieval device into the target tissue area comprises rotating an auger comprising the tissue retrieval device.

26. (canceled)

27. The method of claim 21, wherein:
activating the pressure applying device to bias the tissue collector into the target tissue comprises releasing a spring-loaded deflector; and
releasing the spring-loaded deflector comprises retracting a sleeve from around the spring-loaded deflector.

28. (canceled)

29. The method of claim 21, wherein activating the pressure applying device to bias the tissue collector into the target tissue comprises expanding a body.

30. The method of claim 21, wherein expanding the body comprises inflating a bladder.

31. The method of claim 21, further comprising:
inserting an endoscope comprising a first longitudinal passage into the anatomy of the patient;
inserting an auxiliary scope comprising a second longitudinal passage into the first longitudinal passage; and
inserting the tissue retrieval device into the second longitudinal passage to reach the target tissue.

32. A tissue retrieval device comprising:
an elongate shaft extending along an axis and configured for insertion into an anatomic duct;
a tissue collection device coupled to the elongate shaft and configured to separate tissue from the anatomic duct;
an energization device configured to improve engagement between the tissue collection device and the anatomic duct; and
a control mechanism configured to selectively activate the energization device.

33.-42. (canceled)
